(19) United States
(12) Patent Application Publication
(10) Pub. No.: US 2007/0233137 A1
(43) Pub. Date: Oct. 4, 2007

(54) Tibia cutter

(76) Inventors: Jai-Gon Seo, Seoul (KR); Chong-Bum Kim, Gyeonggi-Do (KR)

Correspondence Address: PARK LAW FIRM, 3255 WILSHIRE BLVD, SUITE 1110, LOS ANGELES, CA, 90010, US

(21) Appl. No.: 11/354,499

(22) Filed: Feb. 15, 2006

(51) Int. Cl. A61F 5/00

(52) U.S. Cl. 606/87

(57) ABSTRACT

A tibia cutter, which enables the artificial tibia insert to be installed at the exact position by indicating information about the fitting position of the artificial tibia insert on the cut section of the tibia, is disclosed. The tibia cutter comprises a body including a bone cutting slot horizontally formed at the upper portion of the body, a plurality of pinholes horizontally formed under the bone cutting slot of the body, an alignment groove vertically formed at the rear side of the body, and an indication hole formed forward and backward along the bone cutting slot in said bone cutting slot; and an indication pin which is inserted into said indication hole to indicate information about the fitting position of artificial tibia insert on the cut section of the tibia that is cut by electric saw.

### BACKGROUND OF THE INVENTION

1. Field of the Invention

The present invention relates to a tibia cutter and a base plate, and more specifically to a tibia cutter and a base plate in which the artificial tibia insert can be installed at the exact position in artificial knee joint replacement surgery by indicating information about the installation position of the artificial tibia insert on the cut section of the tibia.

2. Description of the Related Art

Knee arthroplasty, for replacing a knee joint damaged or deformed due to congenital deformation, traumatic injuries, diseases, or degenerative arthritis, etc. with an artificial knee joint, is widely performed.

We will take a brief look at such knee arthroplasty: After cutting the bottom end of femur and the top-end of the tibia to replace the damaged knee joint with an artificial knee joint 10, an artificial femur insert 11 shown in FIG. 1 is fitted to the bottom end of the cut femur, and an artificial tibia insert 12 is fixed to the top-end of the cut tibia. At this time, an artificial polymer insert 13 as a buffer member is inserted and fitted into the top of the artificial tibia insert fixed on the top-end of the tibia for facilitating relative motion by minimizing frictional contact between the artificial femur insert 11 and the artificial tibia insert 12. The artificial femur insert 11 and the artificial tibia insert 12 inserted through such a process of surgery perform the role of an artificial knee joint 10.

Here, we will take a brief look at the method by which a tibia cutter for cutting the top-end of the tibia and an artificial tibia insert are inserted into the top-end of the cut tibia.

FIG. 2a shows a conventional tibia cutter 20 used in the knee arthroplasty. As shown in the drawing, a bone cutting slot 21 is horizontally formed at the upper portion of the body of the tibia cutter 20 to inset an electric saw for cutting the top-end of the tibia, and a plurality of pinholes 23 are horizontally formed under the bone cutting slot 21 of the tibia cutter 20 to insert fixing pins 22 driven into the tibia. And, an alignment groove 24 is vertically formed at the rear side of the body of the tibia cutter 20 to insert an alignment shaft 31 of a tibia crus alignment device 30.

How the tibia cutter 20 in such a configuration is used will be described with reference to FIG. 2b. As shown in FIG. 2b, after aligning the tibia crus in accordance with the mechanical axis using the tibia crus alignment device 30, the alignment shaft 31 of the tibia crus alignment device 30 is inserted into the alignment groove 24 of the tibia cutter 20. Next, the fixing pins 22 are inserted into the pinholes 23 of the tibia cutter 20 to fix the tibia cutter 20 to the tibia. After that, the tibia crus alignment device 30 is removed and an electric saw is inserted into the bone cutting slot 21 of the tibia cutter 20 to cut the top-end of the tibia.

Subsequently, as shown in FIG. 2c, on the top surface of the cut tibia is fixed a base plate 40 that has an access hole 41 formed in symmetry. Next, a longitudinal installation slot is formed on the top cut section of the tibia using a tibia punch tower 50 and a tibia punch 60, and an artificial tibia insert 12 is installed in this installation slot.

At this time, the position of fixing the base plate 40 fixed on the top-end cut section of the tibia is very important because it will eventually determine the position of fitting the artificial tibia insert 12 that is fitted in the installation slot of the tibia. As shown in FIG. 2d, at the time of fixing the base plate, it should be fixed in such a way that the line of symmetry 32 of the access hole 41 corresponds with the alignment line 32 of the alignment shaft 31 of the tibia crus alignment device 30 that has aligned the tibia crus.

But according to the conventional tibia cutter described above and the surgical method of inserting and fixing an artificial tibia insert to the top-end of the cut tibia, when the top-end of the tibia is cut after removing the tibia crus alignment device 30, there is no information about the section of the cut tibia about the position at which the base plate 30 is to be fixed, namely, the information about the alignment line of the alignment shaft 31 of the tibia crus alignment device 30. Accordingly, there is a problem in that the base plate 40 cannot be fixed on the cut section of the tibia with the line of symmetry of the access hole 41 in accordance with the alignment line of the alignment shaft 31 of the tibia crus alignment device 30, but is fixed in a shifted state to left or right as shown in FIG. 3a, or rotated state as shown in FIG. 3b.

Accordingly, the longitudinal installation slot on the top-end portion of the tibia is formed in an undesired position due to the base plate fixed at an undesired position, and the artificial tibia insert device 12 is inserted and fitted in the installation slot at an undesired position as well. As a result, the relative motion between the artificial tibia insert 12 and the artificial femur insert 11 is not smooth, which causes problems of side effects occurring after surgery and the shortened lifetime of the artificial knee joint.

### SUMMARY OF THE INVENTION

Therefore, it is an object of the present invention to provide a tibia cutter and a base plate for installing an artificial tibia insert at an exact position by indicating the information about the position of fitting the artificial tibia insert on the cut section of the tibia in knee arthroplasty.

In accordance with the present invention, there is provided a tibia cutter comprising a body including a bone cutting slot horizontally formed at the upper portion of the body to insert an electric saw for cutting the tibia top-end, a plurality of pinholes horizontally formed under the bone cutting slot of the body to insert fixing pins to be driven into the tibia, an alignment groove vertically formed at the rear side of the body to insert an alignment shaft of a tibia crus alignment device, and an indication hole formed forward and backward along the bone cutting slot in said bone cutting slot; and an indication pin which is inserted into said indication hole to indicate information about the fitting position of artificial tibia insert on the cut section of the tibia that is cut by electric saw.

In accordance with another aspect of the present invention, there is provided a tibia cutter comprising a body including a bone cutting slot horizontally formed at the upper portion of the body to insert an electric saw for cutting the tibia top-end, a plurality of pinholes horizontally formed under the bone cutting slot of the body to insert fixing pins to be driven into the tibia, an alignment groove vertically formed at the rear side of the body to insert an alignment shaft of a tibia crus alignment device, and an indication hole formed forward and backward along the bone cutting slot in said bone cutting slot; and an indication unit which, while sliding each of the fixing pins driven into said tibia, indicates information about the fixing position of the artificial tibia insert on the cut section of the tibia that is cut by said electric saw.

In accordance with yet another aspect of the present invention, there is provided a base plate in which an access hole is punched in the center in symmetry and which is mounted on the top-end of the cut tibia, wherein the base plate characterized in that a drain is formed forward and backward on the center bottom of said base plate to insert an indication pin smeared with GV dye.

### BRIEF DESCRIPTION OF THE DRAWINGS

Other objects and aspects of the present invention will become apparent from the following description of embodiments with reference to the accompanying drawings in which:

FIG. 1 is a perspective view of a conventional artificial knee joint for replacing a damaged knee joint;

FIG. 2a is a perspective view of a conventional tibia cutter;

FIG. 2b is a drawing in which a conventional tibia cutter is combined with a conventional tibia crus alignment device that aligns a tibia crus;

FIG. 2c is a drawing for describing the process of forming an installation slot by using a tibia punch tower and tibia punch on a conventional top-end cut section of the tibia;

FIG. 2d is a drawing for describing a preferable fixing position of a base plate fixed on the conventional top-end cut section of the tibia;

FIGS. 3a and 3b are drawings for describing a conventional base plate that is fitted at an undesired position on a top-end cut section of the tibia in the prior art;

FIG. 4 is a perspective view of a tibia cutter according to a first embodiment of the present invention;

FIG. 5 is a drawing showing the forming of a drain as information about the position of fitting an artificial tibia insert on a top-end cut section of the tibia according to the first embodiment of the present invention;

FIG. 6 is a drawing showing how a base plate is installed at a desired position on the cut section of the tibia according to the first embodiment of the present invention;

FIG. 7 is a perspective view of a tibia cutter according to a second embodiment of the present invention;

FIG. 8 is a drawing showing the forming of an indication line as information about the position of fitting an artificial tibia insert on a top-end cut section of the tibia according to the second embodiment of the present invention;

FIG. 9 is a drawing showing how a base plate is installed at a desired position on a tibia cut section according to the second embodiment of the present invention;

FIG. 10 is a perspective view of a tibia cutter according to a third embodiment of the present invention;

FIG. 11 is a perspective view of a tibia cutter according to a fourth embodiment of the present invention;

FIG. 12 is a bottom view of a base plate according to the present invention;

FIGS. 13 and 14 are drawings for describing how to use the base plate according to a fifth embodiment of the present invention.

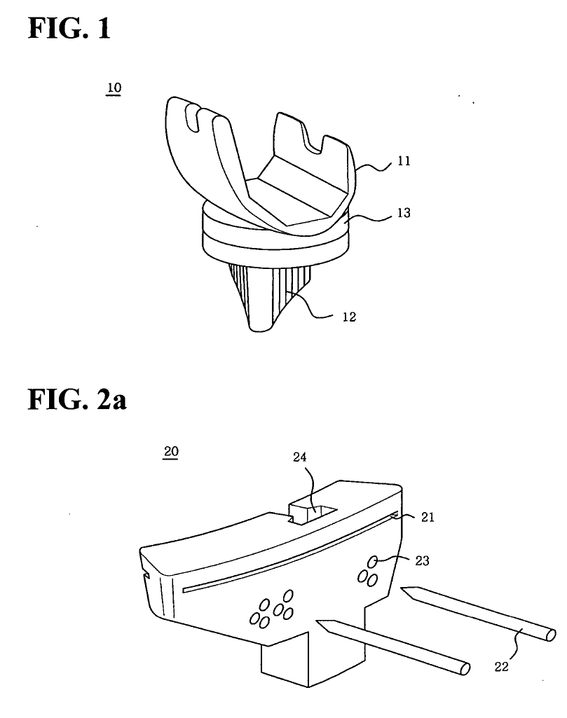

### DESCRIPTION OF THE PREFERRED EMBODIMENTS

Below will be described in detail embodiments according to the present invention with reference to accompanying drawings. The same reference symbol is given as necessary to the component that is the same as the conventional one; and the following the first to fourth embodiments relate to the tibia cutter, and the fifth embodiment relates to the base plate.

### First Embodiment

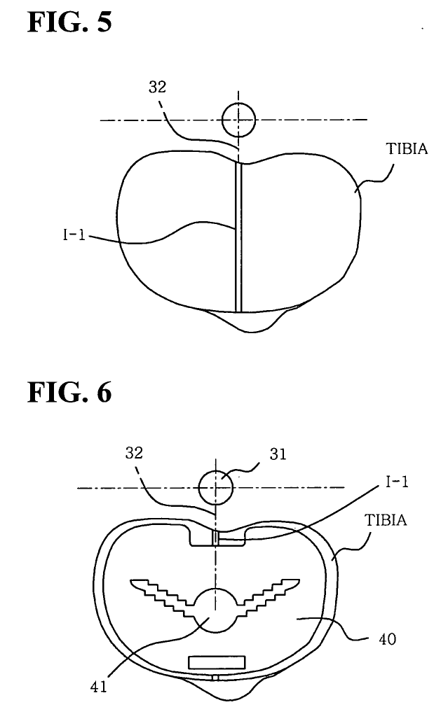

FIGS. 4 to 6 are drawings for describing the first embodiment of a tibia cutter according to the present invention. Specifically, FIG. 4 is a perspective view of a tibia cutter according to a first embodiment of the present invention, FIG. 5 is a drawing showing the forming of a drain as information about the position of fitting an artificial tibia insert on a top-end cut section of the tibia according to the first embodiment of the present invention, and FIG. 6 is a drawing showing how a base plate is installed at a desired position on the cut section of the tibia according to the first embodiment of the present invention.

As shown in FIG. 4, the body of the tibia cutter 120 according to this embodiment comprises a bone cutting slot 121, a plurality of pinholes 123, an alignment groove 124 and an indication hole 125. The bone cutting slot 121 is horizontally formed at the upper portion of the body of the tibia cutter 20 to insert an electric saw for cutting the top-end of the tibia, and a plurality of pinholes 123 are horizontally formed under the bone cutting slot 121 of the tibia cutter 20 to insert fixing pins to be driven into the tibia, and the alignment groove 124 is vertically formed at the rear side of the body of the tibia cutter 20 to insert an alignment shaft 31 of the tibia crus alignment device 30.

The indication hole 125, into which an indication pin 126 for indicating information about the installation position of the artificial tibia insert 12 on the section of the tibia that is cut by the electric saw is inserted, is formed forward and backward along the bone cutting slot 121.

Here, a preferred example of the indication hole 125 is shown in FIG. 4. Referring to this drawing, the indication hole 125 is formed forward and backward along the bone cutting slot 121 at the position perpendicular to the alignment groove 124, and the cross section of this indication hole 125 is a circular shape formed roundly facing the lower side and upper side of the bone cutting slot 121. And the indication pin 126 is preferably a drill bit 126 capable of drilling.

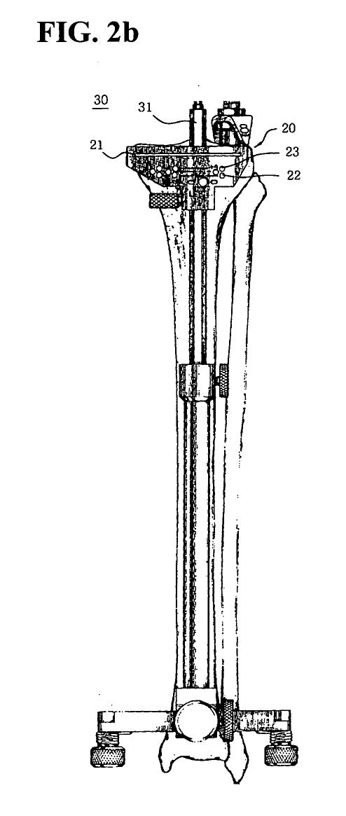

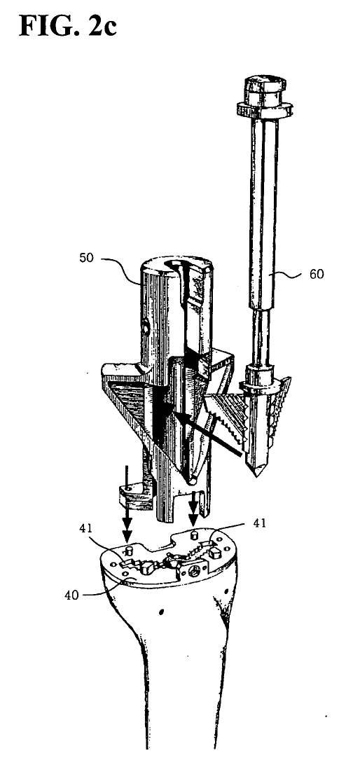

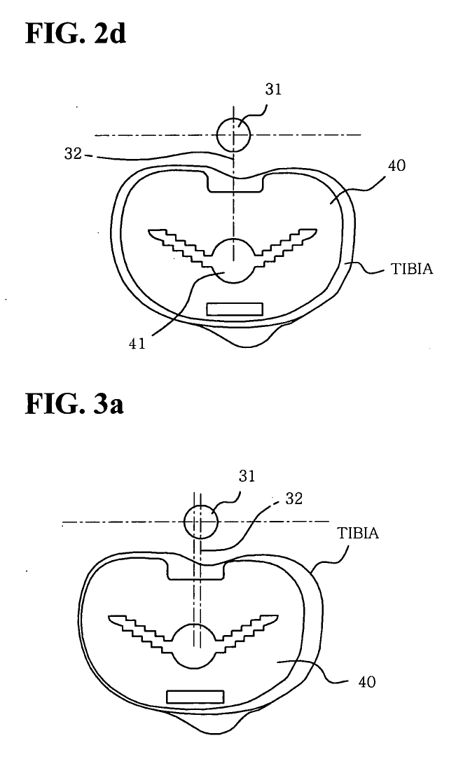

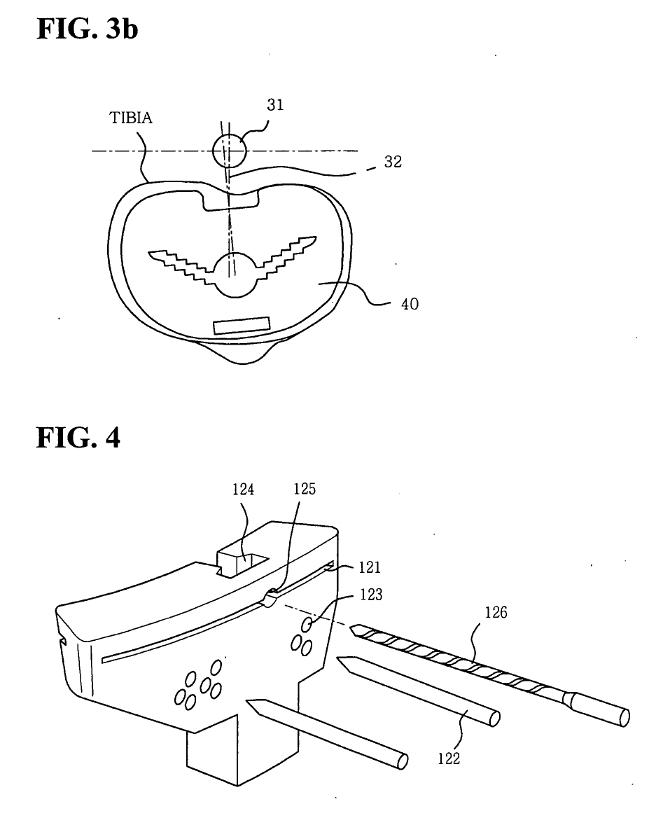

The surgery method of cutting the tibia by using the tibia cutter according to this embodiment is substantially identical with the conventional one described in FIG. 2b. First, after aligning the tibia crus in accordance with the mechanical axis using the tibia crus alignment device 30, insert the alignment shaft 31 of the tibia crus alignment device 30 into the alignment groove 124 of the tibia cutter. Next, fix the tibia cutter to the tibia by inserting each of the fixing pins 122 into each of the pinholes 123 on both sides of the tibia cutter to be driven into the tibia. Then, remove the tibia crus alignment device 30, and insert the drill bit 126 into the indication hole 125 having a circular cross section formed along the bone cutting slot 121 for drilling of the tibia.

Next, insert the electric saw into the bone cutting hole 121 to cut the top-end of the tibia, then, as shown in FIG. 5, a drain I-1 is formed on the top-end cut section of the tibia in accordance with the alignment line 32 of the alignment shaft 31 of the tibia crus alignment device 30 that has aligned the tibia crus.

After that, as shown in FIG. 6, with the access hole 41 of the base plate 40 in symmetry with respect to the drain I-1, fix the base plate 40 on the top-end cut section of the tibia. Next, as shown in FIG. 2c, form a longitudinal installation slot on the top-end portion of the tibia using the tibia punch tower 50 and the tibia punch 60, and after removing the base plate 40, install the artificial tibia insert 12 in the installation slot.

According to the tibia cutter of this embodiment, because a drain I-1, as information about the fitting position of the artificial tibia insert on the cut section of the tibia that is cut by an electric saw, is formed in accordance with the alignment line of the alignment shaft of the tibia crus alignment device on the top-end cut section of the tibia, it is possible to fit the base plate at the exact position of the top-end cut section of the tibia, and accordingly it is possible to install the artificial tibia insert at the exact position of the tibia.

### Second Embodiment

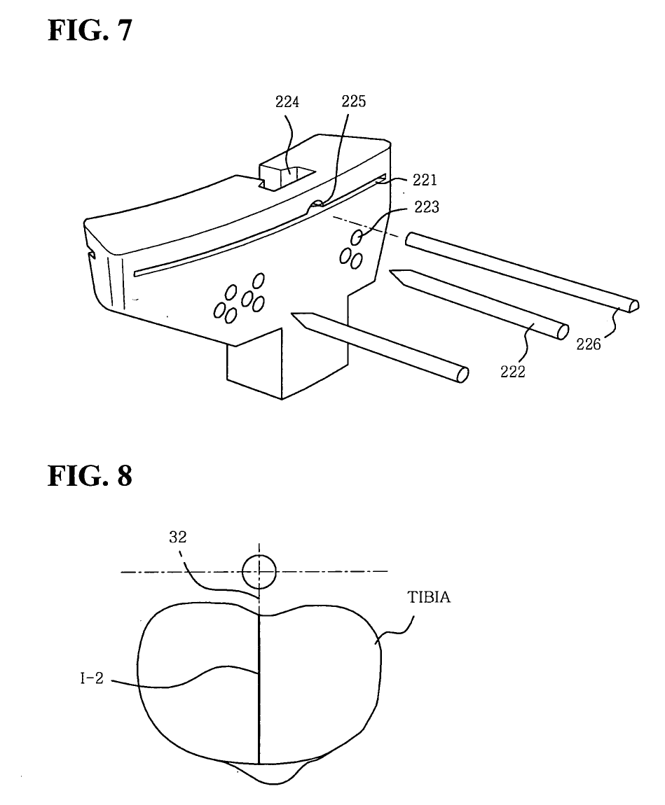

As the second embodiment of the present invention, another preferred embodiment in which the indication hole of the tibia cutter is provided in another shape is shown in FIG. 7. Referring to FIG. 7, an indication hole 225 is formed forward and backward along a bone cutting hole 221 at a position perpendicular to an alignment groove 224, and furthermore the indication hole 225 has a semi-circular cross section shape and the indication pin 226 has a shape corresponding to that of the indication hole 225. The bottom side of the indication hole 225 is in contact with the bottom surface of the bone cutting slot 224.

To use the tibia cutter configured like above, first, as shown in FIG. 2b, align the tibia crus in accordance with the mechanical axis using the tibia crus alignment device 30. Next, insert the alignment shaft 31 of the tibia crus alignment device 30 into the alignment groove 224 of the tibia cutter, and insert each of the fixing pins 222 into each of the alignment grooves 224 of the tibia cutter so as to fix the tibia cutter to tibia. Subsequently, remove the tibia crus alignment device 30, and then insert an electric saw into the bone cutting slot 221 to cut the top-end of the tibia.

Subsequently, insert the indication pin 226 smeared with GV dye into the indication hole 225, then the indication pin 226 advances in contact with the top surface of the top-end cut section of the tibia that is cut by the electric saw and smears the top-end cut section of the tibia with GV dye. Accordingly, as shown in FIG. 8, a line I-2 smeared with GV dye is formed on the top-end cut section of the tibia in accordance with the alignment line of the alignment shaft of the tibia crus alignment device that has aligned the tibia crus.

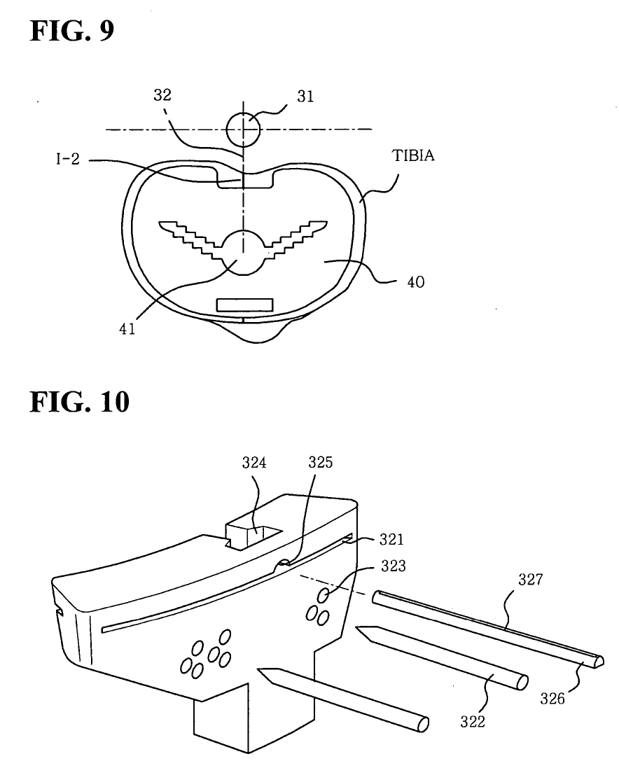

In this embodiment, the line I-2 on the top-end cut section of the tibia smeared with GV dye becomes information about the fitting position of the artificial tibia insert. Therefore, as shown in FIG. 9, after fixing the base plate 40 on the top-end cut section of the tibia in symmetry with the access hole of the base plate 40 with respect to the line I-2 smeared with GV, form a longitudinal installation slot on the top-end cut section of the tibia using the tibia punch tower 50 and the tibia punch 60 described in FIG. 2c. And remove the base plate and install the artificial tibia insert 12 in the installation slot, then the artificial tibia insert can be installed at the exact position of the tibia.

### Third Embodiment

Since the tibia cutter according to the third embodiment is substantially similar to the tibia cutter according to the second embodiment, only the differences will be described.

As shown in FIG. 10, the tibia cutter according to this embodiment is characterized in that a marker hole 327 is formed in the lengthwise center of the indication pin 326. And, the shape of the indication hole 225 into which the indication pin 226 is inserted is identical with the one in the second embodiment. We will take a look at the surgery method: Like the second embodiment, insert an electric saw into the bone cutting slot 321 to cut the top-end of the tibia and insert the indication pin 326 into the indication hole 325. Next, put the indication pin 326 on the top-end cut section of the tibia, and pass a marker through the marker hole 327 formed on the indication pin 326 to draw a line. Accordingly, as shown in FIG. 8, a line, which is in accordance with the alignment line of the alignment shaft of the tibia crus alignment device that has aligned the tibia crus, is formed on the top-end cut section of the tibia.

In this embodiment, the line on the top-end cut section of the tibia marked with the marker becomes information about the fitting position of the artificial tibia insert, and accordingly the artificial tibia insert can be installed at the exact position of the tibia. The surgery method after this is omitted as it is the same with the second embodiment.

### Fourth Embodiment

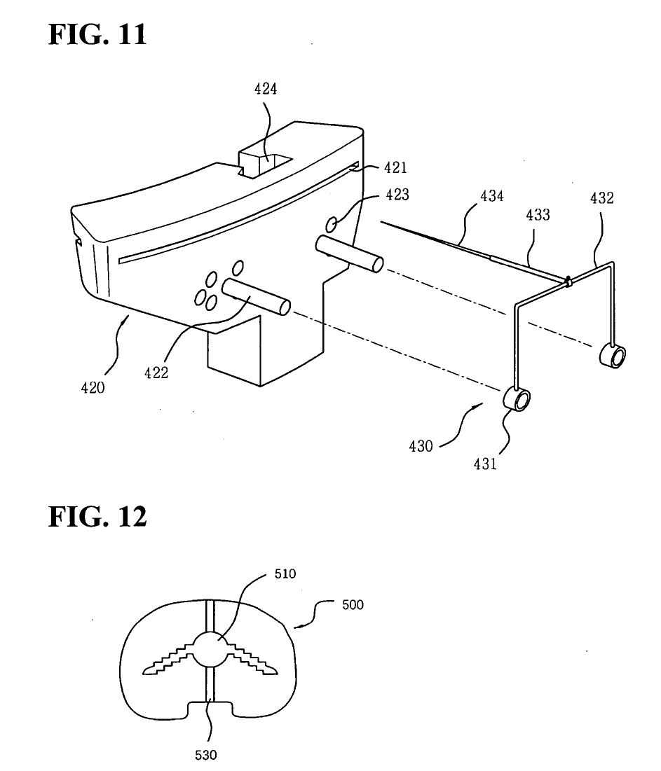

FIG. 11 shows a tibia cutter according to the fourth embodiment of the present invention.

Referring to FIG. 11, the tibia cutter according to this embodiment comprises a tibia cutter body 420 including a bone cutting slot 421 horizontally formed at the upper portion of the body of the tibia cutter to insert the electric saw for cutting the tibia top-end, and a plurality of pinholes 423 horizontally formed under the bone cutting slot 421 of the body of the tibia cutter to insert the fixing pins 422 to be driven into the tibia, and an alignment groove 424 vertically formed at the rear side of the body of the tibia cutter to insert an alignment shaft 31 of the tibia crus alignment device 30; and an indication unit 430 that indicates, while sliding each of the fixing pins 422 driven into the tibia, information about the fitting position of the artificial tibia insert on the cut section of the tibia that is cut by an electric saw.

The body 420 of the tibia cutter according to this embodiment is identical with the tibia cutter of the conventional configuration shown in FIG. 2a, and is characterized by further comprising the indication unit 430 that indicates information about the fitting position of the artificial tibia insert on the cut section of the tibia that is cut by an electric saw while sliding each of the fixing pins 422 driven into the tibia.

Preferably, the indication unit 430 includes holders 431, a link 432 and a marker rod 433.

Each of the holders 431 is slidably assembled to each of the fixing pins 422 driven into the tibia, and the link 432 can be bent so as to connect two holders 431. Also, it is preferred that the holders 431 and the link 432 are formed in one unit.

One end of the marker 433 is slidably installed to the link 432 and the other end has an insert hole formed for the marker 434 to be inserted. For example, in the case of right-hand screw, the marker rod 433 can be fixed to the screw by normal rotation and separated by reverse rotation, and the slope with respect to the link can be selectively adjusted for fixing and separation.

We will see the surgery method of cutting the tibia by using a tibia cutter according to this embodiment configured like above. The tibia crus is aligned in agreement of the mechanical axis by using the tibia crus alignment device 30, and the alignment shaft 31 of the tibia crus alignment device 30 is inserted into the alignment groove 424 of the tibia cutter. Next, the fixing pins 422 are inserted into the pinholes 423 of the tibia cutter to be driven into the tibia so as to fix the tibia cutter to the tibia. At this time, the holders 431 of the indication unit 430 are stuck onto the fixing pins 422 driven into the tibia. And, after the marker rod 433 is moved left and right along the link 432 such that the marker 434 is positioned on the alignment line 32 of the alignment shaft 31 of the tibia crus alignment device 30, the marker rod 433 is fixed to the link 432.

Next, after the tibia crus alignment device 30 is removed and the tibia top-end is cut by electric saw, the holders 431 of the indication unit 430 that have position adjustment finished are stuck onto the fixing pins 422 again and advanced. Then, the marker 433 will indicate on the top-end cut section of the tibia a line that is in accordance with the alignment line 32 of the alignment shaft 31 of the tibia crus alignment device 30.

The line indicated like above becomes information about the fitting position of the artificial tibia insert. After forming an installation slot in symmetry with this line, the artificial tibia insert is installed. Accordingly, it is possible to install the artificial tibia insert at the exact position of the tibia.

### Fifth Embodiment

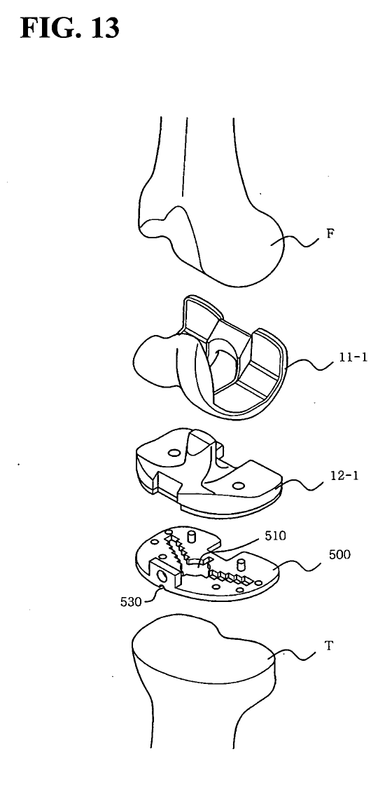

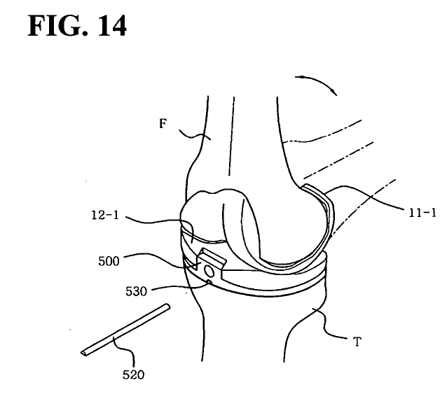

The fifth embodiment according to the present invention relates to a base plate. Specifically, FIG. 12 is a bottom view of a base plate according to the present invention, and FIGS. 13 and 14 are drawings for describing how to use the base plate according to a fifth embodiment of the present invention.

As shown in the drawings, a base plate. 500 according to this embodiment is mounted on the top-end portion of the tibia in which the access hole 510 is punched in the center in symmetry. On the center bottom of the base plate 500 is formed forward and backward a drain into which an indication pin 520 smeared with GV dye is inserted.

In FIG. 13, between tibia T with the top-end cut and femur F with the bottom end cut, there are a test artificial femur insert 11-1, a test artificial tibia insert 12-1 and the base plate 500 in sequence. The test artificial femur insert 11-1, test artificial tibia insert 12-1 and base plate 500 are joined each other, and are temporarily placed between femur F and tibia T, as shown in FIG. 14.

After the test artificial femur insert 11-1, test artificial tibia insert 12-1 and base plate 500 are placed between femur F and tibia T like above, the femur T and the tibia F are moved to and fro in a stretched state and bent state along the arrow direction. When the femur is moved to and fro until the stretching motion and bending motion become smooth, the positions of the test artificial femur insert 11-1, test artificial tibia insert 12-1 and base plate 500 that are joined each other are corrected little by little. In this process, the positions of the artificial femur insert 11 and the artificial tibia insert 12 are exactly set.

When the positions of the test artificial femur insert 11-1, test artificial tibia insert 12-1 and base plate 500 that are joined each other are set, the indication pin 520 smeared with GV dye as shown is inserted/separated into/from the drain 530 that is formed on the bottom of the base plate 500. Then, on the top-end section of the cut tibia is formed a line I-2 as shown in FIG. 8. Since the line like above is a line showing the positions of installation of the artificial femur insert 11 and the artificial tibia insert 12 that are to be replaced, it eventually becomes the line that is practically in accordance with the alignment line 32.

When a line is formed like above, disassemble the test artificial femur insert 11-1, test artificial tibia insert 12-1 and base plate 500 that are joined each other, and mount the base plate 500 on the top-end portion of the tibia. At this time, fit the base plate 500 such that the symmetry line of the access hole 510 is coincide with the line I-2 indicated on the top-end portion of the tibia.

When the base plate 500 is fitted at the top-end of the tibia, form a longitudinal installation slot on the top-end portion of the tibia using the tibia punch tower 50 and the tibia punch 60. And, when the artificial tibia insert 12 is installed after removing the base plate 500, the artificial tibia insert 12 can be installed at the exact position of the tibia.

According to the tibia cutter and base plate of the present invention, it is possible to install an artificial tibia insert at the exact position by indicating information about the fitting position of the artificial tibia insert on the cut section of the tibia.

Although the present invention has been described in detail reference to its presently preferred embodiment, it will be understood by those skilled in the art that various modifications and equivalents can be made without departing from the spirit and scope of the present invention, as set forth in the appended claims.

What is claimed is:

1. A tibia cutter comprising:
a body including a bone cutting slot horizontally formed at the upper portion of the body to insert an electric saw for cutting the tibia top-end, a plurality of pinholes horizontally formed under the bone cutting slot of the body to insert fixing pins to be driven into the tibia, an alignment groove vertically formed at the rear side of the body to insert an alignment shaft of a tibia crus alignment device, and an indication hole formed forward and backward along the bone cutting slot in said bone cutting slot; and
an indication pin which is inserted into said indication hole to indicate information about the fitting position of artificial tibia insert on the cut section of the tibia that is cut by electric saw.

2. The tibia cutter according to claim 1, wherein said indication hole has a circular cross section shape formed roundly facing the bottom side and top side of said bone cutting slot.

3. The tibia cutter according to claim 1, wherein said indication hole has a semi-circular cross section shape formed roundly the top surface of said bone cutting slot, and the bottom side thereof is in contact with the bottom surface of said bone cutting slot.

4. The tibia cutter according to claim 1, wherein said indication hole is formed forward and backward along the bone cutting slot at a position perpendicular to said alignment groove.

5. The tibia cutter according to claim 4, wherein said indication hole has a circular cross section shape formed roundly facing the bottom side and top side of said bone cutting slot.

6. The tibia cutter according to claim 4, wherein said indication hole has a semi-circular cross section shape formed roundly the top surface of said bone cutting slot, and the bottom side thereof is in contact with the bottom surface of said bone cutting slot.

7. The tibia cutter according to claim 1, wherein a marker hole is formed in the lengthwise center of said indication pin.

8. A tibia cutter comprising:
a body including a bone cutting slot horizontally formed at the upper portion of the body to insert an electric saw for cutting the tibia top-end, a plurality of pinholes horizontally formed under the bone cutting slot of the body to insert fixing pins to be driven into the tibia, an alignment groove vertically formed at the rear side of the body to insert an alignment shaft of a tibia crus alignment device, and an indication hole formed forward and backward along the bone cutting slot in said bone cutting slot; and
an indication unit which, while sliding each of the fixing pins driven into said tibia, indicates information about the fixing position of the artificial tibia insert on the cut section of the tibia that is cut by said electric saw.

9. The tibia cutter according to claim 8, wherein said indication unit comprises holders which are slidably joined to each of the fixing pins driven into said tibia, a link which connects said holders, and a marker rod, one end of which is slidably connected to said link and into the other end of which is inserted a marker.

10. A base plate in which an access hole is punched in the center in symmetry and which is mounted on the top-end of the cut tibia,
wherein the base plate characterized in that a drain is formed forward and backward on the center bottom of said base plate to insert an indication pin smeared with GV dye.
